(19) United States
(12) Patent Application Publication
(10) Pub. No.: US 2008/0300564 A1
(43) Pub. Date: Dec. 4, 2008

(54) Absorbent Product

(75) Inventors: Maria Bogren, Askim (SE); Carin Hakansson, Billdal (SE); Kent Hermansson, Vastra Frolunda (SE); Elisabeth Boissier, Vallda (SE); Patrik Andersson, Billdal (SE)

Correspondence Address: DRINKER BIDDLE & REATH (DC), 1500 K STREET, N.W., SUITE 1100, WASHINGTON, DC, 20005-1209, US

(73) Assignee: SCA HYGIENE PRODUCTS AB, GOTEBORG (SE)

(21) Appl. No.: 12/097,664

(22) PCT Filed: Dec. 16, 2005
(86) PCT No.: PCT/SE2005/001961

§ 371 Date: Jun. 16, 2008

(51) Int. Cl. A61F 13/15

(52) U.S. Cl. 604/367; 604/378; 604/385.23; 604/385.01

(57) ABSTRACT

An absorbent product having a rear part, a front part and a crotch part in its longitudinal direction, including a liquid permeable perforated top sheet facing the wearer, a liquid impermeable back sheet facing away from the wearer, and an absorbent structure positioned between the top sheet and the back sheet, and whereby longitudinally extending textile-like edges are positioned on both longitudinal sides of the top sheet, whereby the rear part of the absorbent structure is rounded in the form of at least three different radii. Hereby, it is possible to adjust the shape of the absorbent structure so that more absorbent material is put in the product.

### CROSS-REFERENCE TO PRIOR APPLICATION

This application claims priority to Application No. PCT/SE2005/001961 filed Dec. 16, 2005, which is incorporated by reference herein.

### FIELD OF THE INVENTION

The invention refers to a small-sized absorbent product, especially an incontinence protection, a sanitary napkin or a panty liner including an apertured liquid permeable top sheet facing the wearer, a liquid impermeable back sheet facing away from the wearer, and an absorbent structure positioned between the top sheet and the back sheet, and optionally a liquid distribution layer between the top sheet and the absorbent structure, wherein the top sheet and the back sheet are sealed together at the periphery of the product thereby forming an edge sealing, and whereby longitudinally extending textile-like edges are positioned on both longitudinal sides of the top sheet.

### BACKGROUND OF THE INVENTION

In order to reduce material waste when manufacturing absorbent products such as incontinence protections, sanitary napkins or panty liners, it is beneficial if the different parts of the product have sizes that are adapted for their purpose. With regard to the absorbent structure, it is desirable that the absorbent structure has enough capacity to absorb all liquid that is given off from the wearer, so that any leakage does not occur. Further, since absorbent material, particularly superabsorbent polymer material (SAP), is expensive, it is desirable that the absorbent structure is as small and efficient as possible, i.e., so that the product is designed in a way allowing as much absorbent material as possible to be used. Hence, it is also important that the relationship between top sheet, back sheet, absorbent structure and optionally used liquid distribution layers, such as a high loft layer or an acquisition layer, is optimized. Thus, many different parameters need to be considered to provide an optimally sized, and economically produced absorbent product in large-scale production. The demands are even higher when dealing with small-sized products, since leakage risks are higher, and the efficiency of the product must be enhanced compared to larger products.

U.S. Pat. No. 3,805,790 discloses an absorbent article such as a feminine napkin having rounded edges. The purpose of this disclosure is to provide a feminine napkin having an anatomical and comfortable form. Especially, the concave form of the crotch area is discussed.

SE-A-9904200 discloses an absorbent article, such as a feminine napkin for wearing beneath underwear with narrow rear section. The rear part of the article includes a reinforced part and shape for preventing leakage and visibility when worn with underwear having a narrow rear section.

Accordingly, it is known to elaborate with the shape of an absorbent product in order to obtain advantages with respect to leakage and comfort. However, the focus of the present invention is to optimize the absorption capacity of a small-sized absorbent product.

Still further, when dealing with a product having textile-like edges, which mainly are provided to give comfort and reduce chafing between the skin of the wearer and the top sheet, the liquid transport efficiency from the top sheet to the absorbent structure may be reduced since parts of the top sheet are covered by the textile-like edges. Hence it is even more important to optimize the absorption capacity for a small-sized absorbent product having textile-like edges.

Moreover, when trying to optimize the use of material in an absorbent product, the costs for the material that is used, particularly with the regard to the superabsorbent material, must be considered.

Thus, it is the object of the invention to provide a small-sized absorbent product having optimized absorption capacity, thereby solving the problems posed above.

### SUMMARY

Surprisingly, the inventors of the present invention have shown that this object is fulfilled by a small-sized absorbent product of claim 1, wherein the rear part of the absorbent structure is rounded in the form of at least three different radii. Hereby, it is possible to adjust the shape of the absorbent structure so that more absorbent material is put in the product. Further, the absorption capacity is increased and thus the risk for leakage is reduced. Also, since an absorbent structure having this form will lie closer to the edge sealing of the product, the “inner product space” will be more filled and as a result the product edges will become stiffer. Thus, the contact between the absorbent structure and the top sheet and between the top sheet and the wearer will be better, resulting in a more efficient absorption.

A drawback with a better contact between the product and the wearer could be that any moisture that is formed at the skin of the wearer can not be transported away, particularly at the textile-like edges. However, by providing apertured textile-like edges, this problem can be avoided. Moisture can then be transported away from the skin of the wearer, since the “airiness” and “breathability” of the material is increased.

In a preferred embodiment the first radius (r1) is in the interval from 10 to 40 mm, preferably 20-30 mm, the second radius (r2) is in the interval from 20 to 70 mm, preferably 35-55 mm, and the third radius (r3) is in the interval from 50 to 120 mm, preferably 70-100 mm, whereby r1<r2<r3. The relationship between the magnitude of the various chosen radii should be such that it fits the specific product in question (thus the specific radius size must be chosen to fit the product in question). Further, the set of radii that is chosen should be such that the absorbent structure can fill as much as possible of the product in which it is contained. In order to do this, it is often adequate that the relative relationship between (a) r1 and r2 and (b) r2 and r3, respectively, are essentially equal, so that the set of radii defines a rounding that is as continuous as possible. Moreover, more than three different radii can be used. However, this raises higher demands on the cutting equipment, particularly when the absorbent structure that is cut is small (as for a small, size optimized panty liner). Three radii have shown to be a good balance between optimization of absorption capacity and material waste on the one hand and technical difficulties on the other hand.

In a preferred embodiment, the absorbent product in question has an asymmetrical shape in that it is least wide in the crotch part, and that the front part is laterally broader than the rear part (see FIG. 1). In this case, it is even further important that the rear part shows optimized absorption properties, since it is smaller than for conventionally designed products. Thus, the use of an absorbent structure including at least three different radii in the rear part is even further important.

In another preferred embodiment, the outer edge of the absorbent product is rounded in the form of at least three different radii (R1, R2, R3) whereby R1<R2<R3. Normally, R1, R2 and R3 have similar values as r1, r2, r3. However, what is important is that the interrelationship between the radii for the absorbent structure and the absorbent product optimizes the content of absorption material in the product. Hereby, the rounded absorbent structure and the product are adapted to each other, so that the amount of absorbent material that is included in the product with relation to the product size is maximized.

Normally, this can lead to a certain material waste, since parts of the absorbent structure is cut away. However, this possible material waste is balanced by the benefits that are achieved by the present invention with regard to absorption capacity.

By the present invention, the absorbent capacity in relation to the product size is increased by at least 4%, more preferably by at least 8%, most preferably by at least 15% compared to conventional solutions (i.e., an absorbent structure being 4, 8, 15% longer than normal can be used by cutting it according to the invention). Further, the absorbent capacity in relation to the product area is increased by at least 3%, preferably, at least 5%, more preferably, by at least 10%, and most preferably, by at least 20% compared to conventional solutions (i.e., an absorbent structure having a surface area being 3, 5, 10, 20% larger can be used by cutting it according to the invention).

Since the wetting point (the position on the product that is in contact with the liquid providing position/part of the body of the wearer) can differ in longitudinal position up to about 7 cm between different users and different situations for each user, it is important that small products have an absorbent structure that is as long as possible, in order to reduce the risk for leakage. By improving the length as disclosed in the present invention, this problem is reduced.

Also, since the ratio of the surface area of the absorbent structure compared to the surface area of the product of a panty liner product in accordance with the invention is approximately 54% (about 41 cm² divided with about 76 cm²), this shows that it is especially important to optimize the absorption capacity for a small absorbent product. Thus, in a preferred embodiment, the ratio of the surface area of the absorbent structure and the surface area of the absorbent product is higher than 0.50, preferably, higher than 0.54, and more preferably, higher than 0.58.

In still another preferred embodiment, no essentially straight parts are present in the part of the rear edge of the absorbent structure that is rounded by at least three different radii. Hereby, the shape of the rear edge of the absorbent structure is further optimized.

Moreover, in a preferred embodiment, the product has a longitudinal length that is in the interval from 13-22 cm, preferably, smaller than 15 cm, and a lateral width, at the least wide position of the crotch part, that is in the interval from 4 to 6.5 cm, preferably smaller than 5 cm.

For a small-sized absorbent product, the absorbent structure needs to be formed so that it is positioned as close as possible to the product edge sealing. Alternatively, as much as possible of the product should be filled with absorbent material to maximize the absorbent capacity. For a small-sized product, where the margins are smaller, and where large-scale production put high demands on manufacturing efficiency, cutting techniques for product and/or absorbent structure can also be a bottle-neck.

One important aspect of the invention is that the product of the invention has a high absorption capacity in relation to its size. In order to achieve this advantage, some properties should be shown by the product.

To start with, the liquid inlet materials (top sheet+liquid distribution layer) should be able to take care of a relatively large volume of liquid. This is achieved by using a top sheet material having funnel-shaped three-dimensional apertures, and by providing a bulky material, such as a high loft layer, beneath the top sheet. The three-dimensional structure of the perforated material together with the high loft layer gives a high free volume (void volume) that quickly can take care of the liquid. The funnel-shaped apertures will also “collect” the liquid initially so that it does not flow off the surface. The film of the top sheet material can also be pre-perforated with small apertures, so that the liquid also partly can be taken in between the large apertures.

Secondly, the inlet materials should be able to keep the liquid that has been taken up by the product during use. This is achieved by using the film of the top sheet material as a barrier against rewetting, i.e., to make it difficult for the liquid to return to the skin of the wearer. The SAP of the absorbent core has also, of course, a major contribution to keeping the absorbed liquid in the product. The three-dimensional structure of the top sheet and liquid distribution layer, preferably high loft layer, also makes the distance between the skin of the wearer and the core of the product longer, so that the product is experienced as drier.

Moreover, as a third point, the inlet materials need to provide a dry surface for the comfort of the wearer. This is achieved by ultra-sonically welding the top sheet material and the liquid distribution layer, preferably, high loft layer. Hereby, the top sheet material and liquid distribution layer can be properly drained.

Further, as a fourth point, the inlet materials should be soft and airy for the comfort of the wearer. This is achieved by that the material for the textile edges is made in a soft nonwoven for providing good comfort, and by that it includes perforations for being “airier” and for giving a good inlet.

Thus, the invention is further directed to an absorbent product wherein the product has at least one of the following characteristics (in accordance with the experimental results shown in the example section): (i) an acquisition time for a first inlet of 5 ml synthetic urine that is below 6 s, preferably, equal to or less than 5 s, and an acquisition time for a second inlet of 5 ml synthetic urine that is below 8 s, preferably, equal to or below 4 s; (ii) a DORUP (retention) value for a dosage of 7 ml synthetic urine that is below 1 g, preferably, below 0.5 g and more preferably, equal to or below 0.2 g; or (iii) a Rothwell (Absorption capacity) value of more than 45 g, preferably, more than 55 g.

### BRIEF DESCRIPTION OF THE DRAWINGS

FIG. 1 illustrates a top plan view of a panty liner having asymmetric shape of the invention including apertured textile-like edges. Three different radii of the rear end of the product are shown.

FIG. 2 illustrates a top plan view of another panty liner of the invention including apertured textile-like edges.

FIG. 3 shows a cross-sectional view of various layers of an absorbent product of the invention.

FIG. 4-6 show experimental diagrams referring to the Example section of the invention.

FIG. 7 shows the rear end of an absorbent product and its absorbent core of the present invention each having three different radii.

### DETAILED DESCRIPTION OF THE PREFERRED EMBODIMENTS

The present invention pertains to an absorbent product. By an “absorbent product” is meant a product such as an incontinence protection, a sanitary napkin and a panty liner. Typically, a small sized absorbent product has a length that is in the interval from 13 to 22 cm, and a width that is in the interval from 4 to 6 cm. The following definitions are provided below for further clarity regarding features of the present invention.

By “open area” is meant the percentage of the surface of a material that is composed of apertures and perforations.

By a “longitudinal” direction or “machine direction” is meant the direction along the length of the absorbent product, i.e., from the rear to the front of the product (or vice versa), and by “lateral” direction or “cross-direction” is meant the direction from side edge to side edge of the product, i.e., across the width of the product.

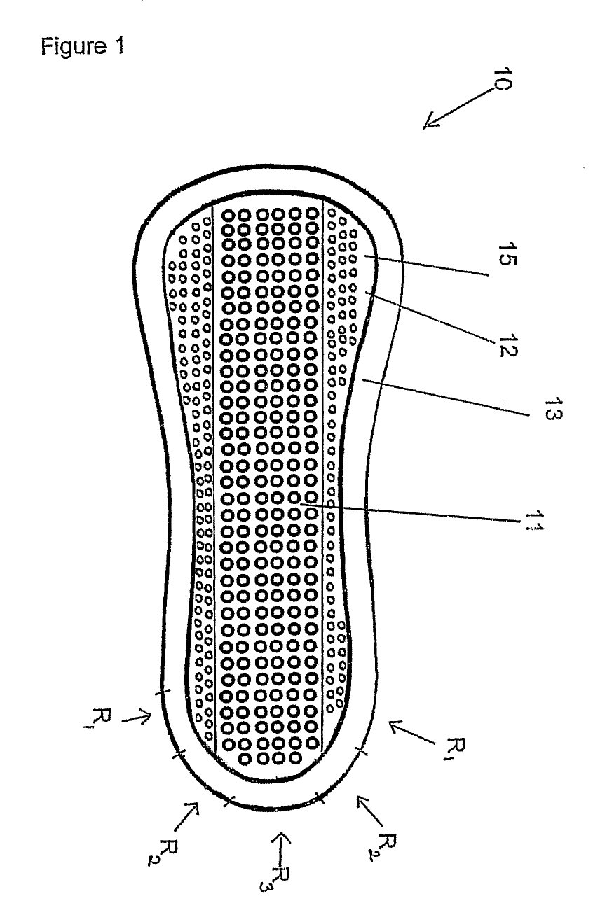

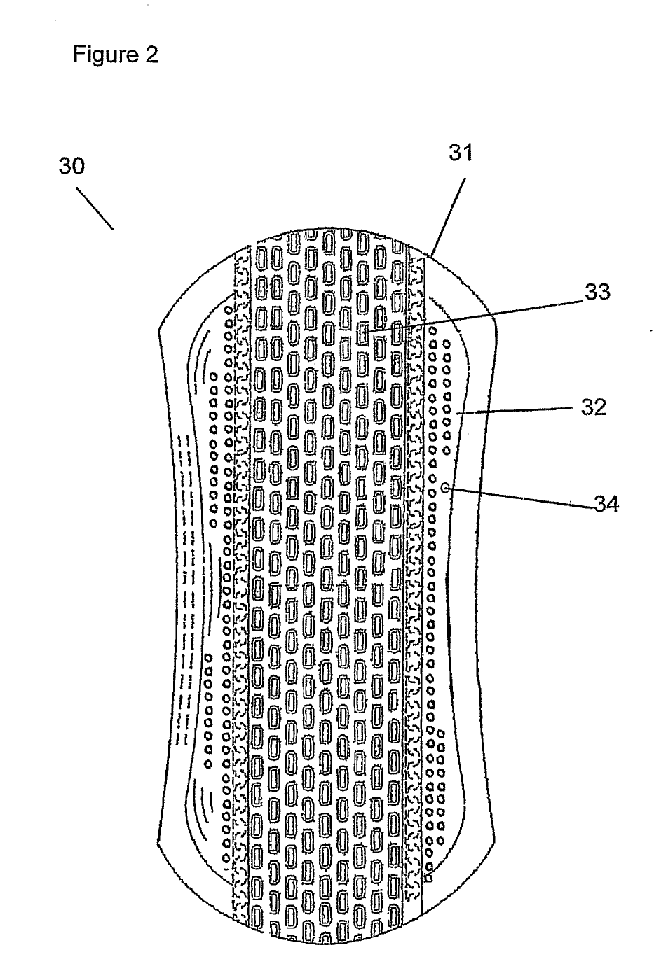

FIGS. 1 and 2 disclose absorbent products in the form of a pantyliner (10) and a pad (30) of the invention. As can be seen the products are equipped with an apertured top sheet (11, 33). At the longitudinal edges of the top sheet, textile-like edges (12, 32) are provided. Preferably, the textile-like edges are apertured (15, 34). The density of apertures in the textile edges will normally be in the interval from 4-250/cm², preferably, from 50-120/cm². Further, the textile-like edges can be equipped with an embossed pattern, in order to add function or for design purposes. At the periphery of the product, an edge sealing (13, 31) can be seen, which seals the various layers of the product. In a preferred embodiment, the edge sealing includes a pattern of discrete welding points, e.g., created by ultrasonic welding. Further (not shown), the top sheet preferably includes small perforations in addition to the larger apertures. Also, it is shown that the product can be cut in three different radii (R1, R2, R3) in order to optimize the fit of the rear edge of the absorbent structure.

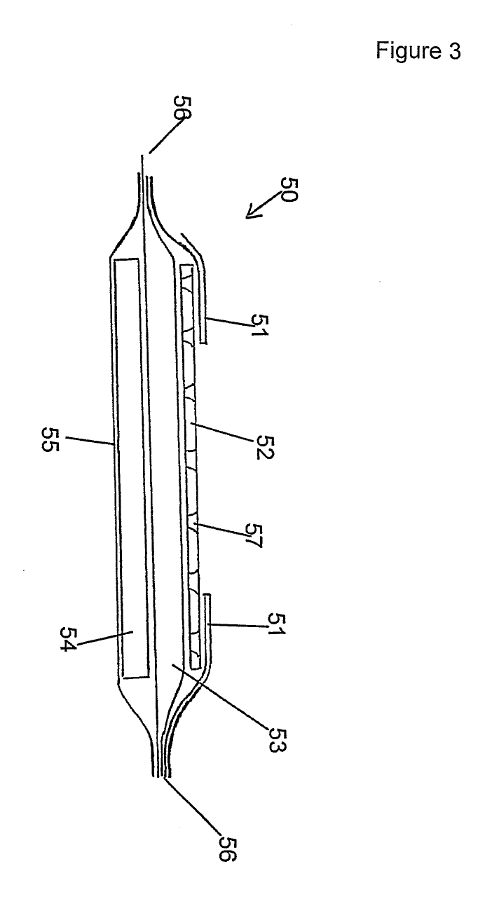

Turning to FIG. 3, a principal drawing of the various layers of the product (50) can be seen. Starting from the top (intended to be closest to the skin of the wearer at use) the textile-like edges (51) can be seen, which are positioned at the longitudinal edges of the top sheet (52). The textile-like edges can be fastened to the top sheet by way of embossing the textile-like edges, by ultrasonic bonding, by using an adhesive, such as a hotmelt, or a combination of fastening devices. Beneath the top sheet an optional liquid distribution layer (53) can be positioned. In a preferred embodiment, the liquid distribution layer is a high loft layer. Beneath the liquid distribution layer, or directly beneath the top sheet (in case no liquid distribution layer is present), the absorbent structure (54) is positioned. For example, the absorbent structure is a pressure-bonded airlaid core including superabsorbent polymers. In a preferred embodiment, in order to provide a high absorption capacity, which is necessary for a small-sized product of this type, the absorbent structure includes about 50-60% SAP. Beneath the absorbent structure, a back sheet (55) is provided. For example, the back sheet, is a plastic film. Preferably, the plastic film is breathable. At the outside of the back sheet a release paper is positioned which is fastened to the back sheet e.g., by glue strings or by any other conventional devices. An edge sealing (56), binding the textile edges, e.g., by way of ultra-sonic welding, the liquid distribution layer and the back sheet at the longitudinal edges can also be seen. Reference numeral 57 refers to a funnel-shaped apertures according to a preferred embodiment.

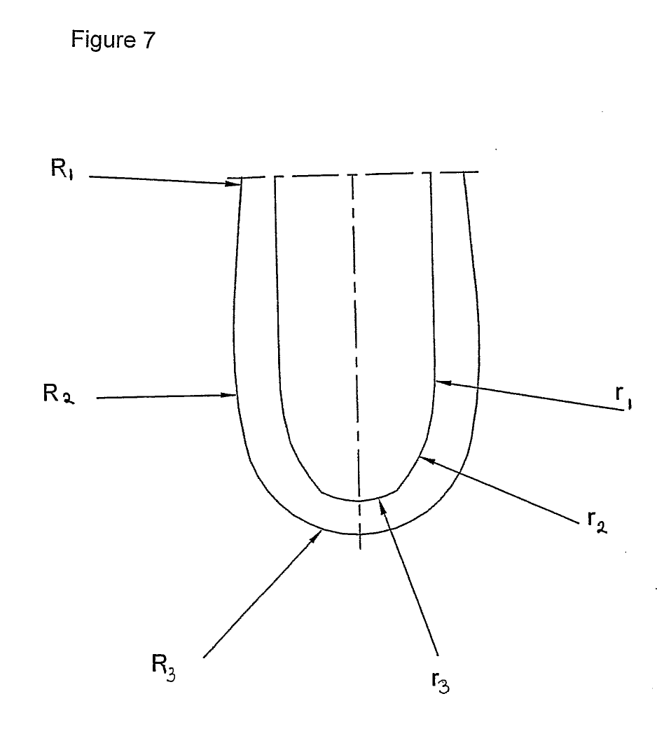

FIG. 7 discloses a product of the invention wherein the different radii of the absorbent structure (r1, r2, r3) and the radii of the absorbent product (R1, R2, R3) are shown. Here, it can be seen that since the rear edge of the absorbent structure is rounded it can extend further to the rear end of the product, and thereby more absorption material can be filled into the product to increase the absorption capacity. Also, the absorbent product can be rounded by three different radii to optimize the fit between the rounded rear edge of the absorbent structure and the inner side of the product.

The cutting of the absorbent structure to the desired radii-shape is normally performed by a mechanical cutting device, a so called “punch”. If a mat-formed absorbent core is used, the form of the structure is normally provided during the mat-forming process by using a form.

The present invention is mainly directed to absorbent products such as an incontinence protection, a sanitary napkin or a panty liner. Each product includes a rear part, a front part and a crotch part positioned there between.

The product can be hour-glass shaped (wherein the rear part and front part essentially equally wide measured in the cross-direction, and the crotch part is less wide than the front or rear part) or it can be asymmetrically shaped or it can have any other shape that is appropriate for products of this type. By asymmetrical can be meant that the product is least wide in the crotch part of the product (measured in cross-direction) and it is wider in the front part than in the rear part (measured at the widest position of the rear and front part respectively in cross-direction). For instance the width in the crotch part of the product can be from 4-7 cm, especially from 4.5 to 5 cm, for example about 4.7 cm. The width at the widest position of the front part can be from 5 to 8 cm, especially from 6-7 cm, for example about 6.5 cm. The width at the widest position of the rear part can be from 4.5 to 6.5 cm, especially from 5 to 6 cm, for example 5.3 cm.

In one preferred embodiment, the product of the invention is a panty liner having a length in the interval from 13 to 22 cm, preferably, of about 15.2 cm and a width at the crotch part of the product in the interval from 4 to 6 cm, preferably, of about 4.7 cm. The top sheet material is a three-dimensional pre-perforated film which is equipped with further apertures, typically 7-9 apertures per row. At the edges of the top sheet a textile edge, preferably apertured, is provided that is composed of nonwoven material. Preferably, a high loft layer is included as a liquid distribution layer. Further, the absorbent core is provided in the form of a roll-material including about 40-60% SAP. The edges of the product including the liquid distribution layer are sealed by way of ultra sonic bonding. The product further includes a back sheet, facing away from the wearer.

In another preferred embodiment, the invention refers to a panty liner including a carded nonwoven as a top sheet material. At the edges of the top sheet a textile edge, preferably apertured, is provided that is composed of nonwoven material. The liquid distribution layer is composed of an airlaid material and the absorbent core includes pulp and SAP. The edges of the product including the liquid distribution layer are sealed by of ultra sonic bonding. The product further includes a back sheet, facing away from the wearer.

In still another preferred embodiment, the invention refers to a sanitary napkin including an apertured top sheet material. At the edges of the top sheet a textile edge, preferably apertured, is provided that is composed of nonwoven material. Further, the sanitary napkin includes a liquid distribution layer and an absorbent core. The edges of the product including the liquid distribution layer are sealed by way of ultra sonic bonding. The product further includes a back sheet, facing away from the wearer.

In yet another preferred embodiment, the invention refers to an incontinence product or incontinence guard including a liquid pervious top sheet facing the wearer during use, optionally a liquid distribution layer underneath the top sheet, an absorbent core, and a liquid impermeable back sheet facing away from the wearer. Normally, the absorbent core includes a compressed mixed or layered structure of cellulosic fluff pulp and superabsorbent polymers, however further or other material combinations as disclosed below are fully possible. Also, instead of being provided in one layer, the absorbent core may include two separate layers, or more.

The shape of the products of the invention can be hour-glass shaped (i.e., being less wide in the central part compared to the front and rear parts, which are equally wide). Also, the products of the invention can be asymmetrically shaped, whereby the product is less wide in the central part compared to the front and rear parts, and whereby the rear part is less wide than the front part. Other shapes are also fully possible for the products of the invention.

The textile-like edges are preferably made by a soft, skin-friendly material, such as a nonwoven, so that any chafing against the skin of the wearer is reduced as much as possible. For example, the material of the textile-like edges can be a carded nonwoven. Preferably, the textile-like edges are apertured. Further, the apertures of the textile edges can be positioned in one or more than one longitudinal or lateral rows, or they can be positioned in a longitudinally extended row. Further the apertures can have varying sizes and forms. They can also be concentrated (zoned) to one or more zones of the textile edge, such as close to the crotch area or the expected wetting point, so that the function of the apertures is concentrated to positions where it is especially desired.

The liquid-permeable top sheet is preferably made of a material showing properties like dryness and softness at use of the absorbent product, as this sheet lies against the body of the wearer. It is desired, that the sheet has a soft and textile-like surface, which remains dry also at repeated wettings. The top sheet may for example be composed of nonwoven material with a soft and smooth surface, such as for example a spunbond made of polypropylene fibers. In order to keep the surface closest to the skin of the wearer dry, a hydrophobic nonwoven-material may be used, which has holes, so that openings are formed in the material, which openings are greater than the cavities between the fibers of the material. In this way, fluid may be led down through the holed openings in the top sheet to the underlying absorption core. Other examples of material in the top sheet may for example be holed plastic films, such as, for example, a holed polyethylene film. The top sheet may be connected to the underlying back sheet and to the absorption core by, for example, glue, ultra-sonic bonding or through some kind of thermal bonding. Preferably, the top sheet is an apertured nonwoven, having an aperture density of 3-15, preferably, 6-12 and more preferably, 7-9 apertures/cm².

It is desirable that the caliper value of the top sheet material is as high as possible, since a high caliper value has a beneficial effect on the acquisition. However, in order to not affect the softness of the material negatively, a balance needs to be obtained, and thus a caliper value in the interval from 1.3 to 1.7 mm, preferably, about 1.5 mm, has shown to be adequate.

The liquid-impermeable back sheet includes a flexible material, preferably a thin plastic film of PE (polyethylene), PP (polypropylene), a polyester, or some other kind of suitable material, such as a hydrophobic nonwoven-layer or a laminate of a thin film and a nonwoven material. These types of laminates are often used in order to achieve a soft and a textile-like surface of the back sheet. In order to accomplish an airier and comfortable product it is also possible to use breathable back sheets, which prevents fluid from coming out of the absorbent product, but that allows moisture to be ventilated. These breathable back sheets may be composed of single material layers, or of laminates of, for example, blown or moulded polyethylene films, which have been laminated with, for example, a nonwoven layer of spunbond or of spunbond-meltblown-spunbond (SMS).

The absorbent structure is typically built up by one or more layers of cellulose fibers, for example cellulose fluff pulp. Other materials, which may be used, are for example absorbing nonwoven material, foam material, synthetic fiber materials or peat. In addition to cellulose fibers or other absorbing materials, the absorbent structure may also includes superabsorbent material, so called SAP (super absorbent polymers), that is material in the form of fibers, particles, granula, film or the like, which material has the ability to absorb fluid corresponding to several times the weight of the superabsorbent material. The superabsorbent material binds the fluid and forms a fluid-containing gel. Moreover, the absorbent structure may include binders, form-stabilizing components or the like. The absorbent structure may be chemically or physically treated in order to change the absorption properties. For instance, it is possible to provide an absorbent layer with compressed regions and/or being compressed in the entire layer(s) in order to control the fluid flow in the absorbent body. It is also possible to enclose the absorbent layer(s) in an envelope of for example tissue material. For example, the absorbent structure is an airlaid, pressure-bonded structure including 30-80%, preferably about 40-60%, more preferably 50-60% SAP.

Typically, the absorbent structure has in its longitudinal direction an outstretched form, and may for example be essentially rectangular, T-shaped or hourglass-shaped. An hourglass-shaped absorbent body is wider in the front and rear parts than in the crotch part, in order to provide an efficient fluid absorption simultaneously as the design facilitates the product to form and to close around the user, thereby giving a better fit around the legs. In a preferred embodiment of the present invention, the absorbent structure has essentially straight and parallel longitudinal edges. The rear and front edges are rounded in order to fit the shape of the product. Preferably, the rear and front edges are rounded in the form of at least three different radii.

Also, the absorbent structure can be provided in the form of a roll-material, whereby the material typically is provided with a high compression, or the structure can be formed by way of air-laying technique.

In yet another embodiment, the absorbent structure is equipped with a wicking layer, which wicking layer has the purpose to spread fluid towards the front part of the absorbent structure. Moreover, the wicking layer does not necessarily need to cover the whole absorbent structure, but should preferably cover at least the part of the absorbent structure being in the front part of the casing, more preferably, the part being in the front and crotch parts of the casing, and most preferably, the entire absorbent structure.

The wicking layer is of a moisture permeable material, preferably, tissue paper or a hydrophilic non-woven, and functions to disperse the fluid, i.e., urine, passing through the liquid permeable top sheet, preferably, in a direction towards the front part of the diaper. The wicking layer includes small capillaries directing the fluid towards smaller capillaries, due to capillary forces.

Additional liquid distribution layers may be used in the product of the invention, preferably between the absorbent structure and the top sheet. For instance, additional layers improving the properties may be used, such as a transfer layer or various types of fluid-spreading material layers or inserts, so called waddings or high-loft layers. Typically, the liquid distribution layer is a porous, resilient, relatively thick material layer, for example in the form of a fibrous high loft layer, a carded fibrous web, a tow material or other type of bulky and resilient fibrous material having a high momentaneous liquid receiving capacity and which can temporarily store liquid before it is absorbed by the underlying absorbent core. Also, the liquid distribution layer may be in the form of a porous foam material. Also, it may includes two or more material layers. In a preferred embodiment, the liquid distribution layer extends to the side edges of the product, i.e., it has basically the form of the top sheet or the top sheet plus the textile edges. Hereby, advantages with regard to liquid distribution, edge sealing etc, as discussed above, can be achieved. However, the liquid distribution layer can also be designed so that it does not extend into the edge sealings. The transfer layer can be an airlaid layer and it can include SAP.

Further, the product of the invention may be equipped with a release paper that covers the outer side of the back sheet of the product. The release paper is fastened to the back sheet by way of glue strings.

At the periphery of the product, with a width of about 0.5 cm, an edge sealing is provided in order to secure the top sheet, the back sheet and optionally the liquid distribution layer to each other. The edge sealing can be made by way of adhering the layers to each other, by way of embossment, by way of heat welding, by way of ultra sonic bonding, or a combination of these methods. Preferably, ultra-sonic bonding is used. Also, the sealing can be made as a continuous sealing or as discrete point weldings. Preferably, the sealing is made as point weldings, since this provides some advantages with regard to, e.g., hydrophobic character of the welding points as discussed above. Typically, the welding points are at a distance of about 0.1-0.5 mm from each other, preferably, about 0.2 mm. For performing the ultra-sonic bonding, an ultra-sonic bonding equipment is used. When the ultra-sonic bonding is performed, a welding pattern is formed, which welding pattern can have a form that is visually attractive and/or that have technical advantages, such as an improved comfort for the wearer or effects with regard to liquid distribution or improved softness and/or dryness.

In order to prevent fluid to leak out, the absorbent product on the side that is facing the wearer may also be equipped with inner fluid barriers, which are attached in connection to the longitudinal edges. Preferably, the inner barriers are made of an essentially liquid-impermeable material, such as for example a hydrophobic nonwoven or a plastic film, and are formed as a longitudinal path with a first edge being connected to the absorbent product and a second free edge, which is adapted for being in close contact with the user at use of the absorbent product. The second edge is equipped with one or more elastic elements, preferably an elastic thread, which in contracted state contracts the free edge, whereby an upstanding barrier is formed. The inner barrier may be designed as a strip of a single sheet, wherein the free edge is turned down in order to enclose the elastic element to prevent direct contact of the elastic thread to the user. Alternatively, the barrier may be formed of two combined layers, whereby the elastic thread is attached to the edge of the free end between the two layers. In this case, the inner layer of the barrier may be composed of an elongation of the top sheet and the outer layer of an essentially liquid-impermeable material, or the inner and outer layers of the barrier may be composed of one single material strip, which is folded around the elastic thread. Further, the elastics can be provided in the form of foam, e.g., in a band or thread, or the elastics can be provided in any other conventional way.

Further, the product of the invention may be equipped with wings. Also products equipped with other layers than described in this disclosure are also included in the scope of the invention.

### EXAMPLES

In all the attached examples, properties were compared between a panty-liner product according to one embodiment of the invention (here called “Tena”) and various conventional panty-liner products (see table 1 and 2 for data for the compared products). The compared products have structures that are similar to Tena. The “Tena-product” has an asymmetric shape (see FIG. 1 for a principal drawing), the top sheet is a perforated laminate between a nonwoven material and a plastic film having a basis weight of about 40 gsm having apertured top sheet that also includes small perforations. Further, the product includes textile edges, a nonwoven high loft layer (basis weight of about 50 gsm) as liquid distribution layer, an absorbent core of cellulose fibers and SAP (about 50-60%) and a breathable back sheet. The “Tena” product is a small panty liner product, and thus the experimental values should be interpreted with regard to a small panty liner. However, since the results that are shown for the Tena product can be regarded as an effect of using quick inlet materials (top sheet having large apertures, textile edges having apertures, and an absorbent core having a large amount of SAP (about 50-60%)), the analogous results can be expected for other product types that are formed in a similar way by the same principles, but being longer, heavier and/or thicker.

TABLE 1

| | | | Length |
|---|---|---|---|
| Product | Weight (g) | Thickness (mm) | (mm) |
| Tena | 3.4 | 2.9 | 152 |
| Alldays Normal | 2.8 | 2.9 | 150 |
| Always pantiliners Regular (US) | 3.1 | 2.6 | 230 |
| Alldays Small | 1.7 | 2.8 | 142 |
| Libresse Normal | 2.9 | 2.8 | 150 |
| Carefree Original | 2.4 | 3.1 | 160 |
| Kotex Normal | 2.1 | 2.6 | 152 |
| Poise light liners | 3.3 | 2.7 | 165 |
| Alldays Extra Large | 3.9 | 3.0 | 177 |
| Always pantiliners Long (US) | 4.1 | 3.1 | 280 |
| Carefree Maxi Large | 3.0 | 3.0 | 180 |
| Lindor active mini Ultra | 3.8 | 2.3 | 165 |
| Libresse Large | 3.8 | 2.6 | 175 |

TABLE 2

| | | | | | | | |
|---|---|---|---|---|---|---|---|
| | | Carefree | Libresse | Kotex | Alldays | Alldays | Alldays |
| Construction | Tena | Original | Normal | Normal | Small | Normal | Extra Large |

| | | | | | | | |
|---|---|---|---|---|---|---|---|
| Surface | laminate | Thermobound | Carded | Nw | 3D-PE | 3D-PE | 3D-PE |
| | | PP-nw | nw | “Coform” | “cotton- | “cotton- | “cotton-like” |
| | | | | | like” | like” | |
| Drainage | High loft | Thermobound | Airlaid | no | Thermo- | Thermo- | Thermo- |
| | layer | bicomponent | | | bound | bound | bound |
| | | PP/PE | | | PP-nw | PP-nw | PP-nw |
| Absorption | Novathin, | Novathin, | Pulp and | Airlaid | Airlaid | 2-layer | 2-layer |
| material | SAP | airlaid | SAP | pulp- | with SAP- | airlaid | airlaid |
| | | with SAP | | system | fibers | with SAP | with SAP |
| SAP (% of | About | — | About 8 | no | — | About 24 | About 31 |
| product) | 50-60 | | | | | | |

### Example 1

### Acquisition

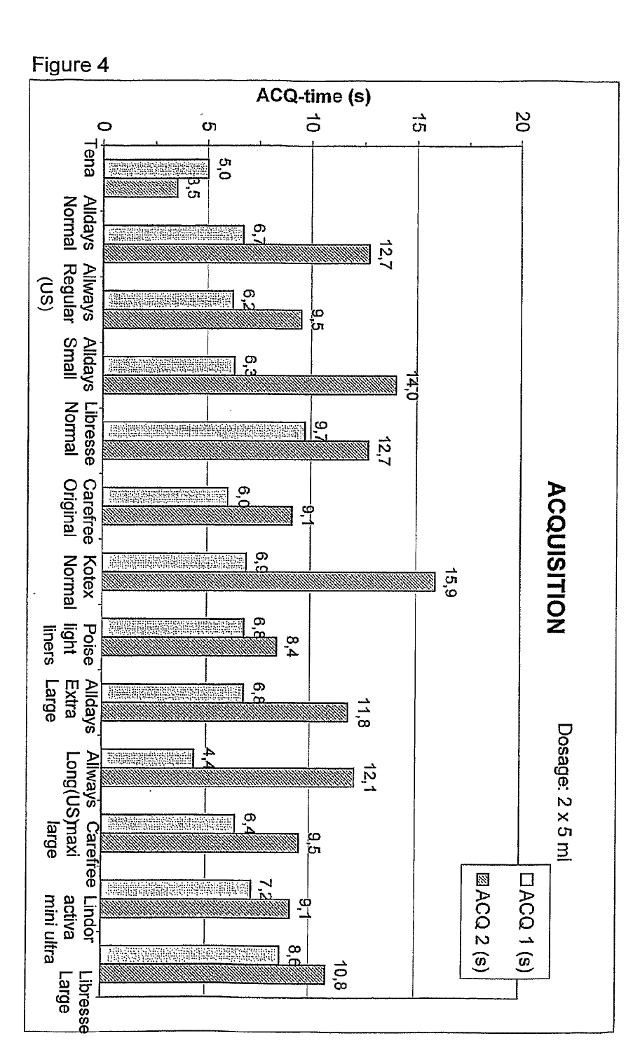

Acquisition properties (acquisition time) were tested (test method no 17-28-14) by exposing the various products for a first inlet of 5 ml synthetic urine (0.9% NaCl in water) followed by a second inlet of 5 ml synthetic urine with an interval of 10 min. The results can be seen in FIG. 4, showing that Tena is significantly faster than almost all the other products for the first inlet, with exception to Always pantiliners Long, and significantly faster than all other products for the second inlet. This shows that the construction of Tena is very capable of quick acquisition of liquid (particularly of urine type), thereby reducing risk for any leakage.

### Example 2

### DORUP (Retention)

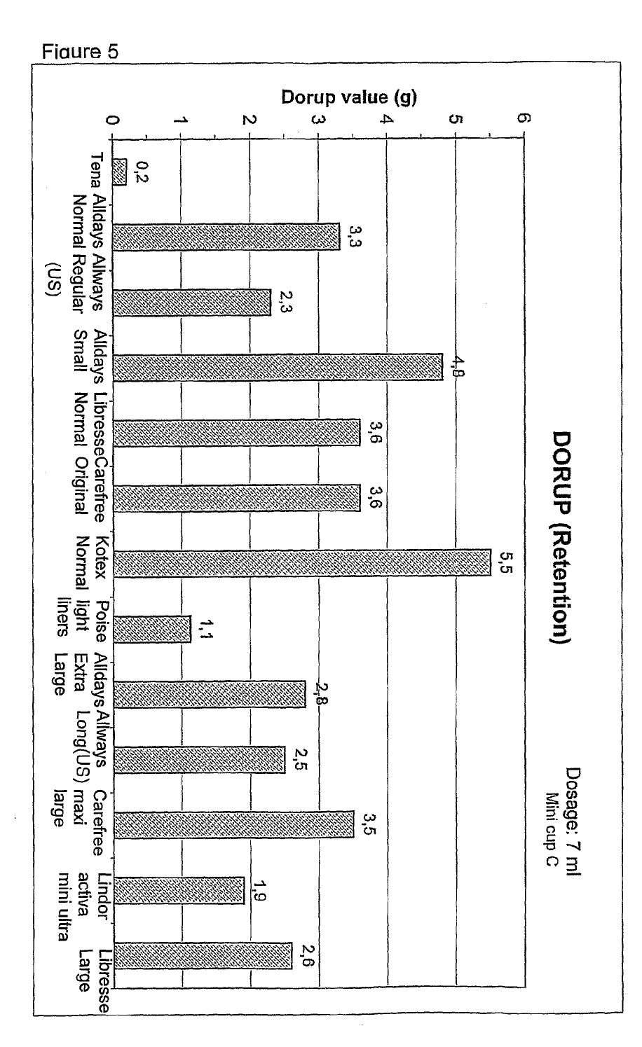

Retention properties were tested by exposing the various products for an inlet of 7 ml liquid (synthetic urine) (see FIG. 5). For more details concerning the DORUP (retention)-experiment, reference is made to U.S. Pat. No. 6,557,398, which is incorporated herein by reference. As can be seen, Tena is significantly drier than all compared products. This shows that the retention properties of Tena is superior to compared products.

### Example 3

### Rothwell (Absorption Capacity)

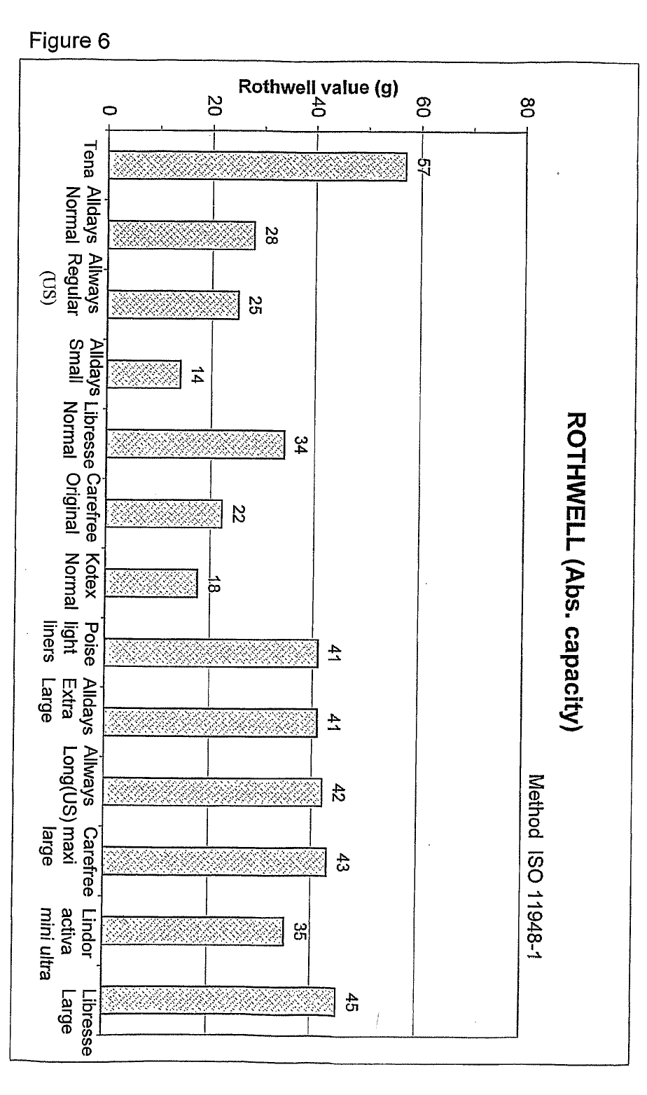

The Rothwell value (g) was tested (Method ISO 11948-1) (see FIG. 6). Tena was shown to have a significantly higher absorption capacity than all compared pantyliners.

1. An absorbent product chosen from an incontinence protection, a sanitary napkin and a panty liner, having a rear part, a front part and a crotch part, comprising:
a liquid permeable perforated top sheet facing the wearer,
a liquid impermeable back sheet facing away from the wearer, and
an absorbent structure positioned between the top sheet and the back sheet, whereby longitudinally extending textile-like edges are positioned on both longitudinal sides of the top sheet,
wherein the rear part of the absorbent structure is rounded in the form of at least three different radii.

2. The absorbent product according to claim 1, wherein a first radius (r1) is in the interval from about 10 to 40 mm, a second radius (r2) is in the interval from about 20 to 70 mm, and a third radius (r3) is in the interval from about 50 to 120 mm, whereby r1<r2<r3.

3. The absorbent product according to claim 1, wherein an outer edge of the absorbent product is rounded in the form of at least three different radii (R1, R2, R3) whereby R1<R2<R3.

4. The absorbent product according to claim 1, wherein the absorbent product has an asymmetrical shape in that it is least wide in the crotch part, and that the front part is laterally broader than the rear part.

5. The absorbent product according to claim 4, wherein the crotch part has a width of about 4.5 to 5.0 cm, the front part a width of about 6.0 to 7.0 cm, and the rear part a width of about 5.0 to 6.0 cm, wherein all values are measured at the widest position of each product part.

6. The absorbent product according to claim 1, wherein the product has an acquisition time for a first inlet of 5 ml synthetic urine that is below 6 s, and an acquisition time for a second inlet of 5 ml synthetic urine that is below 8 s.

7. The absorbent product according to claim 1, wherein the product shows a DORUP (retention) value for a dosage of 7 ml synthetic urine that is below 1 g.

8. The absorbent product according to claim 1, wherein the product shows a Rothwell (Absorption capacity) value of more than 45 g.

9. The absorbent product according to claim 1, wherein the product has a longitudinal length that is in the interval from about 13-22 cm, and a lateral width, at the least wide position of the crotch part, that is in the interval from about 4 to 6.5 cm.

10. The absorbent product according to claim 1, wherein the textile-like edges are perforated.

11. The absorbent product according to claim 1, whereby no essentially straight parts are present in the part of the rear edge of the absorbent structure that is rounded by at least three different radii.

12. The absorbent product according to claim 1, wherein a ratio of the surface area of the absorbent structure and the surface area of the absorbent product is higher than 0.50.

13. The absorbent product of claim 6, wherein the product has an acquisition time for the first inlet of 5 ml synthetic urine that is equal to or less than 5 s, and an acquisition time for a second inlet of 5 ml synthetic urine that is equal to or below 4 s.

14. The absorbent product of claim 7, wherein the DORUP (retention) value for a dosage of 7 ml synthetic urine is below 0.5 g.

15. The absorbent product of claim 7, wherein the DORUP (retention) value for a dosage of 7 ml synthetic urine is below 0.2 g.

16. The absorbent product according to claim 8, wherein the Rothwell (Absorption capacity) value is more than 55 g.

17. The absorbent product according to claim 9, wherein the longitudinal length of the product is smaller than 15 cm and the lateral width of the product is smaller than 5 cm.

18. The absorbent product according to claim 12, wherein the ratio of the surface area of the absorbent structure and the surface area of the absorbent product is higher than 0.54.

19. The absorbent product according to claim 12, wherein the ratio of the surface area of the absorbent structure and the surface area of the absorbent product is higher than 0.58.
